(19) United States
(12) Patent Application Publication
(10) Pub. No.: US 2013/0046357 A1
(43) Pub. Date: Feb. 21, 2013

(54) TISSUE OR NERVE TREATMENT DEVICE AND METHOD

(71) Applicant: Joseph Neev, Laguna Beach, CA (US)

(72) Inventor: Joseph Neev, Laguna Beach, CA (US)

(21) Appl. No.: 13/565,398

(22) Filed: Aug. 2, 2012

Related U.S. Application Data
(63) Continuation-in-part of application No. 12/412,835, filed on Mar. 27, 2009, now abandoned.
(60) Provisional application No. 61/147,148, filed on Jan. 26, 2009.
(60) Provisional application No. 61/140,935, filed on Dec. 27, 2008.
(60) Provisional application No. 61/121,884, filed on Dec. 11, 2008.
(60) Provisional application No. 61/094,375, filed on Sep. 4, 2008.
(60) Provisional application No. 61/093,236, filed on Aug. 29, 2008.
(60) Provisional application No. 61/060,516, filed on Jun. 11, 2008.
(60) Provisional application No. 61/056,150, filed on May 27, 2008.
(60) Provisional application No. 61/048,503, filed on Apr. 28, 2008.
(60) Provisional application No. 61/039,842, filed on Mar. 27, 2008.

(51) Int. Cl. A61N 1/375; A61N 5/067

(52) U.S. Cl. 607/45; 607/89

(57) ABSTRACT

A device and a method for the treatment, modification, imaging, and guiding targeted brain tissue and brain activity are described herein.

### CROSS-REFERENCE TO RELATED APPLICATIONS

The present application is a continuation-in-part to U.S. patent application Ser. No. 12/412,835, filed Mar. 27, 2009, which claims priority to U.S. Provisional Application Ser. No. 61/147,184, filed Jan. 26, 2009, U.S. Provisional Application Ser. No. 61/140,935, filed Dec. 27, 2008, U.S. Provisional Application Ser. No. 61/121,884, filed Dec. 11, 2008, U.S. Provisional Application Ser. No. 61/094,375, filed Sep. 4, 2008, U.S. Provisional Application Ser. No. 61/093,236, filed Aug. 29, 2008, U.S. Provisional Application Ser. No. 61/060,516, filed Jun. 11, 2008, U.S. Provisional Application Ser. No. 61/056,150, filed May 27, 2008, U.S. Provisional Application Ser. No. 61/048,503, filed Apr. 28, 2008, and U.S. Provisional Application Ser. No. 61/039,842, filed Mar. 27, 2008, all of which are incorporated by reference in their entirety.

### BACKGROUND

The invention generally relates to external stimulation, and more particularly to tissue or nerve stimulation.

Under some circumstances it become useful to modify and/change tissue or brain functions or the functioning of at least some brain components inside the human brain. The present invention attempts to solve these problems as well as others.

### SUMMARY OF THE INVENTION

An apparatus and method are provided for treating brain and other targeted tissue. Functional deficiencies, for example hearing impairments, vision impairment, depression or other neural problems may be treated by the methods and devices of the present invention. Other components also provide a device and a method that can be used safely and effectively to treat neurosensors deficiencies (for example, vision impairment or blindness, among other neuro-deficiency or neural-based problems) and hearing impairment.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1 shows a view of the energy or light output coupler design to deliver the stimulating energy to the nerve endings or cochlea.

FIG. 2 shows another possible embodiment of an energy or light source and an output light coupler for delivering light energy for stimulating the hearing nerve along the cochlea.

FIG. 3 is a sectional view of some of the main components of the apparatus for practicing the device and method for nerve stimulations.

FIG. 4 shows a sectional view taken through the artificial neurosensing treatment device that uses light or other energy form to replace natural inputs.

FIG. 5 is shows a view of the device including a microphone, control unit, delivery member, and energy sources.

FIG. 6 shows a sectional view taken through another embodiment of the device, including an OCT for depth measurement and a delivery/imaging coupler.

FIG. 7 shows a block diagram of the elements of one embodiment of the method for treatment neurosensors deficiencies and hearing impairment.

FIG. 8 shows how the device might be used to treat neurosensors deficiency and hearing impairment.

FIG. 9 shows additional exemplary areas of an exemplary brain target and the functional regions associated with said brain regions.

FIG. 10 is a flow chart for a method and devices for modification of tissue and brain functions.

FIG. 11 is a schematic drawing showing another embodiment, where an energy source is directed into the skull and reaches the brain to stimulate blood flow and neural activities.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof

The present invention provides a device and method for treating nerves or neuro-sensors deficiencies and hearing impairments. Other components of the present invention also provide a device and a method that can be used safely and effectively to treat neuro-sensoring deficiencies (for example, vision impairment or blindness, among other neuro-deficiency or neural-based problems) and hearing impairment.

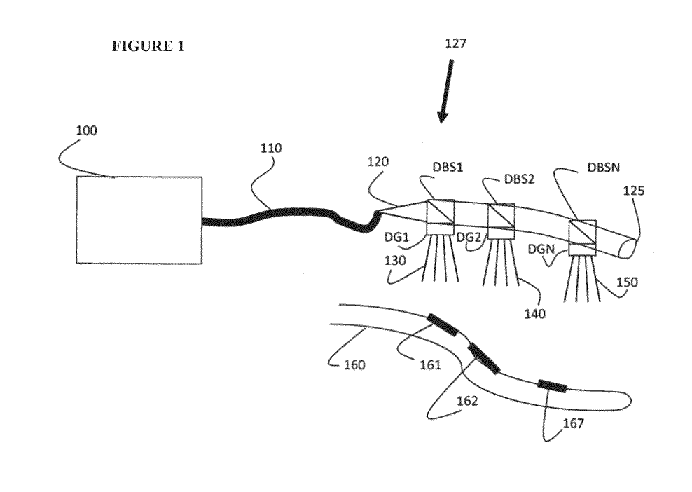

FIG. 1 shows a view of the energy or light output coupler design to delivery the stimulating energy to the nerve endings or cochlea. FIG. 1 shows energy or light from an energy source 100 is coupled to an energy conduit 110 (for example, a hollow waveguide or a single-mode optical fiber, 110). The light form the conduit is expanded through a silica spacer 120 and is optionally focused using a gradient index (GRIN) lens. Different wavelength components of the light in the fiber 110, are then sequentially directed to sequential ports 1, 2, . . . N, by a dichroic beam splitters (DBS), labeled DBS1, DBS2, . . . DBSN, and diffracted using a transmission grating (for example, a grating with 1,000 lines per mm) shown in FIG. 1, by DG1, DG2 . . . DGN, said diffracted gratings are fabricated on the output coupler 125 of the coupler probe 127. The diffraction gratings thus generate frequency-dependent (or color dependent spreading beams, 130, 140 . . . 150, (or Diffracted patterns (DP)-DP1, DP2 . . . DPN). The diffracted patterns are directed towards different cochlear tissue zones corresponding to different frequency components that may be excited in order to generate the neural sensation of sounds in the brain. The Figure also shows the unfolded cochlea 160, and the various excitation zones along the cochlea, 161, 162 . . . 167, etc.

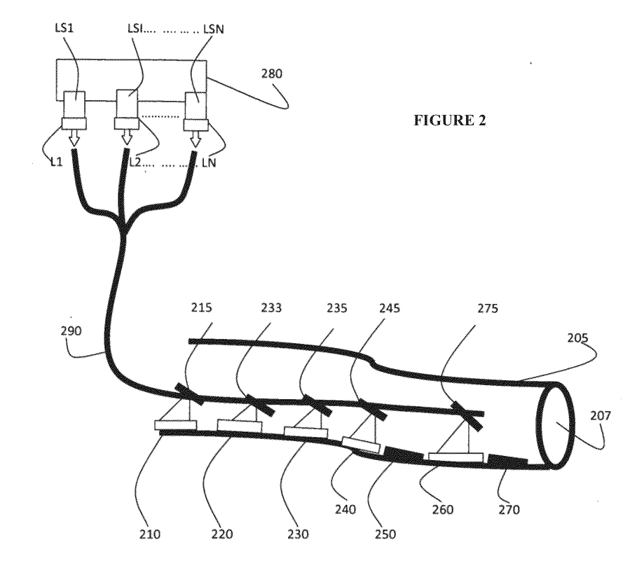

FIG. 2 shows another embodiment and in particular, stimulating the hearing nerve in the cochlea. It shows the design of FIG. 1 repeated at regular length interval along a single output coupler, for example the output coupler 127 described in FIG. 1. As was shown in FIG. 1, the output coupler 127 can be designed to have a beam splitter at repeated intervals in space. Thus part of the light from the source is intercepted at Fiber Beam Splitter 1 (FBS1) 215 and directed out towards a cochlea damaged intervals, for example, interval 220, (or, for example, other damaged cochlea hair at intervals 250, 270 etc.), the remaining of the light (for example, about 96% of the original light) continues towards FBS2 233 where, for example, about 4% of the can again be diverted out towards a new segment of the cochlea—Cochlea Interval 2. The process can then be repeated as need to cover the entire cochlear section (For example, FBS3, and on to FBSN). In addition the feedback or reflected light can be collected to provide continuous 3 dimensional imaging of the cochlear 205 surface and layers below the cochlear surface 207, as provided by the SE method described herein. The figure also shows the energy source 280 that includes, for example, a series of light sources LS1, LS2 . . . LSN. Each light source may also be equipped with its own filter, (for example, a bandwidth filter designed to allow only one color (wavelength) of light, if a broad band (white light) source is used) a coupling lens, L1, L2 . . . LN, where the coupling lenses couple the output light from each source to an optical fiber, said optical fibers from each light source, are then combined to a deliver conduit 290, for example a delivery optical fiber, 290. As described, the light is then split in the fiber beam splitters FBS1...FBSN, said beam splitters may also be dichroic or otherwise enable delivering of a specific color (wavelength) band through each light splitting action. The split light components (carrying the frequency, amplitude and phase information corresponding to the incoming sound frequency that would have normally excite the cochlear hair at a given location (for example, location 210, 220, 230 . . . 270 etc.) can then further split or diffracted to finer spectral component by diffraction gratings and is directed toward the cochlear regions where cochlear hair damage occurred to a larger (for example, regions 250, 270, 220, etc.) or smaller damage extent (for example, regions 210, 230, 240 etc.). If information and imaging of deeper layers of the cochlear tissue is to be obtained, the split or diffracted light groups can be coupled to an OCT and used to obtain three dimensional information.

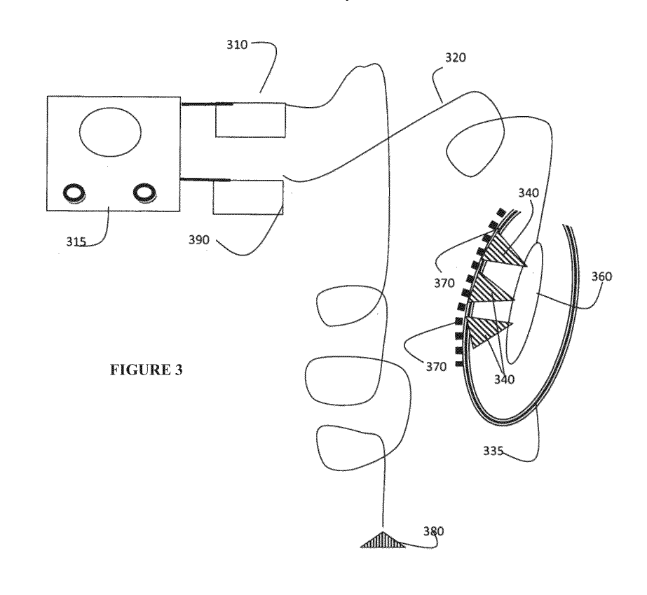

FIG. 3 is a sectional view of some of the main components of the apparatus for practicing the nerve stimulation of the embodiments disclosed herein. FIG. 3 shows the entire apparatus for practicing the embodiments disclosed herein. An energy or light source, 310, for example, a broad band light source is controlled by a controller 315 and coupled to a delivery member, 320. The delivery member (or delivery conduit) 320, may comprise a hollow wave guide, or a fiber, or an electric wire, or other member capable of delivering the energy from the source of energy. The delivery member be contained inside an external member, for example an endoscope or other larger diameter rigid cover capable of being inserted into the targeted tissue or, for example, inside the cochlea.

The deliver member 320 lead to an output coupler 360 that may be constructed as described in FIG. 2. It may be inserted into the cochlea, 330, and be stretched along the entire length the cochlear. From each one of the delivery ports, FBS-1 through FBS-N, a spectral spread will be delivered substantially spread linearly along a segment of the cochlea, 335, and each spectral component 340 may be used and directed to target a certain audio neural ending or a group of neural ending along the cochlea wall 370. By repeating the delivery ports FBS-1 through FBS-N along substantially the entire cochlear length to substantially cover the entire cochlear hearing frequency range. (Alternatively, in cases where only partial hearing loss in only certain sound frequencies, a delivery member can possibly stretch along through only part of the cochlear length, the cochlear portion where hearing losses had occurred). Possibly even unused neural stimulation may also be stimulated for possible useful purposes. The energy source 310 may be couple to a sound interceptor, 380, for example a microphone 380. The sound interceptor may be couple to an analyzer/controller 390. The sound is analyzed and its frequency components (along with their intensity and phase) are then rapidly processed by a processor within the controller/analyzer, 390. The controller analyzer 390 is coupled to the controller 315 to allow generation of the appropriate signal in the energy or light source 320. The information on the incoming sound, from the interceptor or microphone 380, is processed into an output signal that generate intensity and phase of the light components that is being used to stimulate the cochlea component, said cochlear component is known to be capable of responding to the original frequency components of the incoming sound. By sending to the conduit 320 frequency components of the proper wavelength, phase and intensity, AND by using dichroic (i.e. color dependent) beam splitter or fiber beam splitter (FBS) along the delivery conduit as sown in FIG. 3, the proper light wavelengths components corresponding to the correct sound components may be ensured, with the substantially proportional and correct phase and intensity will reach the proper nerve ending along the cochlear (or for that matter any part of the auditory nerve fibers leading to the proper brain hearing centers, that may be chosen to couple to the external system), and allow restoration of hearing in case that there is a damage to hair cilia along the cochlea, or to other components along the hearing chain, for example, the tympanic membrane, the middle ear bones, the hearing canal, the cochlea or some of the auditory hearing nerves.

The same method can be also used to artificially recreate, generate and couple signal from other input systems. For example, a light or vision interceptor 380, (for example, a CCD, video camera or camera recorder), may replace the hearing interceptor, and be used to couple artificial, sight signals, to the visual nerves in the brain or the back of the eye, in order to create vision in the blind or people or animal with other types of vision impairments.

Using the same principals, smell, touch, sexual, or other sense-based stimulator may also be generated. For example, optionally or additionally, anti-depression stimulator may be used. Another embodiment may be, additionally or optionally, hunger suppression in obese people or animals, stimulators of sexual dysfunction in animals or humans. Another embodiment may additionally or optionally use a system as described above for suppression of epileptic seizure, schizophrenia or other nerve or brain malfunctions.

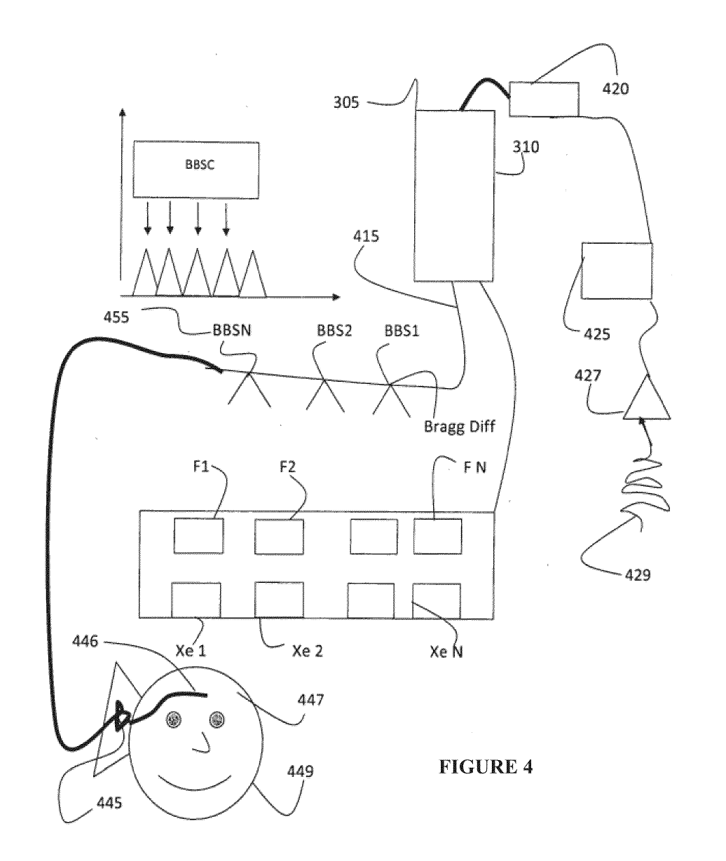

FIG. 4 shows a sectional view taken through the artificial neuro-sensing treatment device that uses light or other energy form to replace natural inputs. Here the energy source 310, can for example, comprise a broad band lamp, for example a xenon lamp with an emission ranging from, for example, from about 400 nm to about 1200 nm. Alternatively, the energy source can comprise, a plurality of LEDs each of different wavelength, or a plurality of laser diodes, each of different wavelength, or a plurality of super luminescence diodes (SLD) each, substantially of different wavelength, alternatively or additionally, the energy source, can comprise a plurality of xenon lamps 417, each with a band-width filters 407, alternatively or additionally, the energy source can comprise other sources of light, electromagnetic energy or other sources of energy.

The energy from the energy source, for example, a plurality of light emitting sources as described above, can be modulated and modified in response to an input signal, 420. The input signal 420 in turn, is modified in response to input from a member 425, capable of analyzing a sound input and providing, phase, amplitude, and frequency information on the arriving sound energy waveform 429. The sound waveform 429 is intercepted by a member 427, capable of detecting said sound energy waveform, for example a microphone 427. The input from the member 425, send the appropriate signal (amplitude, and phase) to each one of the energy source generators, 305, so that said energy source generator provide a corresponding unique optical phase amplitude and wavelength that can be delivered along the energy conduit, for example a fiber, 415.

Thus, in an embodiment, the frequency, phase and amplitude of an input signal, for example, a sound input, are analyzed and translated to a corresponding, energy output generated by the device, for example, the energy output comprises of phase, amplitude, and wavelength components generated by a plurality of light sources, as described above.

FIG. 4 shows the output described above is delivered through a conduit, 415, for example an optical fiber or a hollow waveguide, which intern is inserted into the vicinity of the targeted nerve tissue to be stimulated, 445. The nerve tissue to be stimulated 445, can, for example, comprise a cochlea, or, for example, the hearing nerve 446 in other parts of the brain 447 and inside the skull 449. The source energy can then be launched into a conduit capable of delivering the energy, for example an optical fiber, 415.

The energy, for example optical energy, can then be “decoded by components within the conduit 415, for example, within the optical fiber 415 to allow delivery of different wavelength components (carrying the various sound wave components, 429) to the appropriate target locations within the cochlea 445, or other auditory nerve components 446. This deconstructing of the energy, for example, light energy, into its coded components can be accomplished, for example, in the following way:

A series of dichroic beam splitters, DBS, 455, (for example, DBS1, DBS2 . . . DBSN) can divert only the component of the fiber-delivered optical energy designated to a particular spatial location along the cochlea. The series of dichroic beam splitters, thus allow delivery of specific components of light into different targeted spatial location of the targeted nerve or cochlear tissue. Additionally or alternatively, splitting of the incoming light energy into a desired frequency components is achieved through the use diffraction grating that can be built into the fiber or the GRIN components of the fiber.

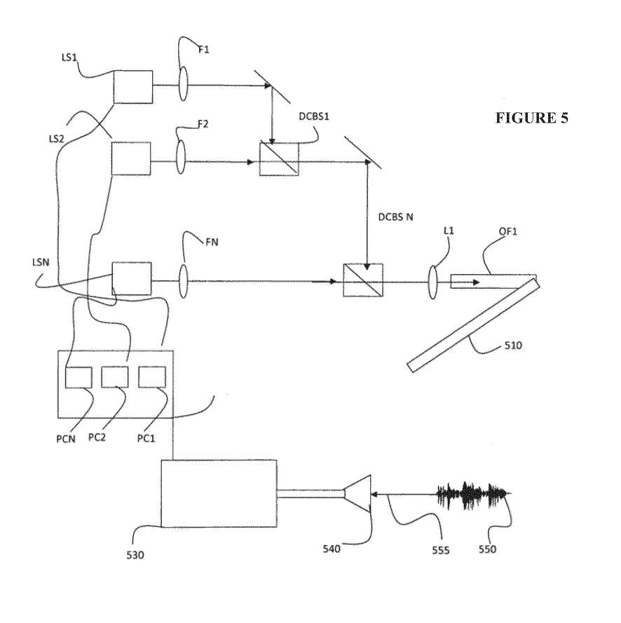

FIG. 5 shows an embodiment of the present invention illustrating how energy or light is coupled to the system. Light form the source LS1, LS2 . . . LSN are combined with Beam Splitters BS1, BS2, . . . BSN, and mirrors (M1, M2 . . . MN) and with lenses (L1, L2, . . . LN) are coupled to a delivery conduit 510, for example, a hollow wave guide or an optical fiber. Optionally, for example if the Light source are broad band, a filter, F1, F2 . . . FN can be used to select a particular wavelength.

The parameters of each light source are controlled by a parameter control electronic signal form a parameter control electronic box (PC1, PC2 . . . PCN). The output parameter control elements PC1 to PCN, are instructed by a processor, 530 what signal to provide each Light source (LS). The processor 530, receives the input signal from a microphone 540, that, in turn, intercept the incoming sound wave (or other external stimuli) 550, propagating towards it in the direction of the arrow 555. The microprocessor, in turn analyze the incoming signal and determine its frequency component content. The frequency components information is used to generate the parameters delivered to each of the energy or light source, to generate the appropriate output light frequency, phase, and amplitude delivered into the energy conduit or fiber 510.

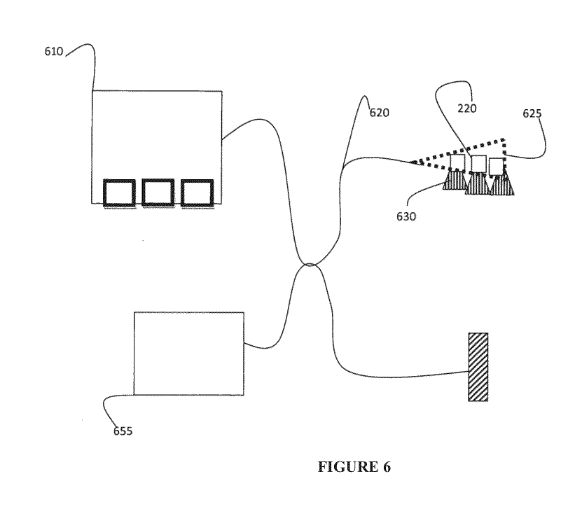

FIG. 6 shows a sectional view taken through the distal end of the Energy delivery member. FIG. 6 shows an energy source 610, for example, a broadband light emanating from an energy conduit, 620, for example an optical fiber 620. The broadband light can be separated into different wavelengths 630, using a lens/grating pair 220 at the ports along the length of the probe (as is also shown in FIG. 6).

In this exemplary configuration light at Port N 625, is deflected by a beam splitter BSN along the conduit length, towards the exit port. A grating lens arrangement can focus each wavelength component onto a different location on the tissue, as shown in FIG. 6.

In addition, reflected light, returned back through the optics and fiber, can then be decoded outside of the ear, using a spectrometer, to form one line of the endoscopic image. Image acquisition can be performed at rates ranging up to 30 kHz. A two-dimensional image may be acquired by rotating or moving the fiber using methods and devices known in the art, such as a stepper motor, a galvanometer, or piezoelectric transducers located at or near the control apparatus.

The sensory or cochlear activating implant can be very small in diameter, for example an optical fiber as small as 100 micrometer or even 50 micrometer can be used. Additionally, the number of neural “pixels” or the resolution of the imaging from backscattered light can be very high, depending on the spectral width of the light source and the ability of the probe to separate out the different wavelength components.

As discussed above, two spectral splitting methods are established: (a) coarse—using, for example, dichroic beam splitter directing encoded colors (with sound, or other sensory input) into the nerves or cochlea, and a fine a FINE spectral splitting using an optical/diffraction grating splitting at the ports (i.e. corresponding optical/grating components described in FIG. 6, placed at each one of the port, i.e. Port 1, 2 . . . N-625).

Additionally, If the light source is configured to be part of the an Optical Coherent (OCT) Interferometer 655, the reflected light can also provide depth information as well as blood flow or movement information through Doppler effects, i.e. Doppler OCT with, for example, blood follow information for the imaged depth. If the OCT is combined with polarization sensitive element, the device and method contemplated herein can also provide information on polarization and birefringence as a function of depth in the target material.

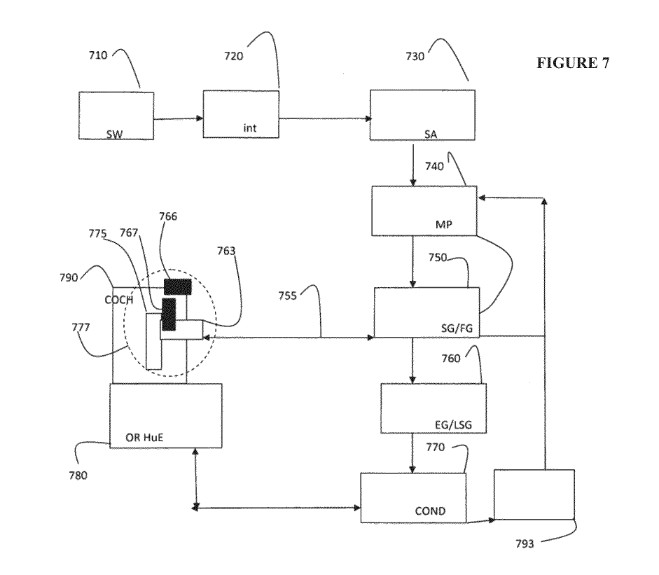

FIG. 7 shows a block diagram of the elements of one embodiment of the method for treatment neurosensors deficiencies and hearing impairment. As shown in FIG. 7, a sound wave (or other external excitation) 710 arrive at and/or is intercepted by a detector 720. The signal is from the interceptor is analyzed by a signal analyzer/processor 730 and further processed by the main processor/computer 740. The main processor/computer 740 control a signal generator 750, that send electronic signals with amplitude, phase and frequency information to an energy source or light source, 760. The light or energy output from the light, laser or energy sources, is coupled to a conduit 770, for example an optical fiber or a hollow wave guide. The optical fiber or other energy conduit 770, enters the organ to be treated, 780, for example the ear, and is then placed in a sufficient proximity to the organ to be stimulated, for example, a nerve or a cochlea 790. The portion of the conduit or optical fiber 770 that is placed close to the cochlea or nerve to be stimulated, is designated the fiber delivery portion (FDP), 775. The FDP, can be designed as described above, with GRIN Lenses, fiber beam splitters, diffraction gratings, lenses, filters or any other optical components as needed.

Alternatively or additionally, the FDP, 775, can be separated from the optical fiber or conduit 770 and form—along with an independent light or energy source, 767, for example a miniature, LED or miniature solid state lasers. This separated, small, energy or light source 767, can be powered by an independent, small, implantable power source, 766, for example a miniature battery. In this case signals with the frequency, amplitude and phase information to modulate the implantable light or energy source 767, can be received wirelessly through a small wireless, implantable receiver, 763. The broken line 755 shows the path of communication between the signal generator 750 and an independent, implantable assembly 777, namely, the set of components, namely the FDP 775, the receiver 763, the power source 766, and the energy or light source 767.

In another embodiment, the implantable assembly 777, or the assembly connected to the external device through the conduit or optical fiber 770, can collect the reflected energy or light and delivery it back to an OCT 793 that proceed to forward the information back to the processor/computer 740 for further analysis and feedback information. (i.e. information such as three dimensional images, depth and morphology information, blood flow, polarization, oxygen levels, temperature, electrical, magnetic, and electromagnetic activities, elastic, and mechanical properties—all of which can be derived from a reflected energy and light that is then collected by the conduit or fiber 770 ad delivered back to the main processor or computer 740. Alternatively or additionally, an implantable, insertable, or swallowed module, 777, may be used to wirelessly, send back the optical information to the main processor or computer 740 for similar further processing and analysis. Alternatively or additionally, the implantable, insertable or swallowed, module 777, may be used to wirelessly send back the signal carrying the information collected from the reflected light or energy, to the signal generator, 750, for an initial amplification, and only then, after the initial amplification (and possible initial processing, for example, filtering) in 750, the signal is sent back to the main processor/computer, 740 for analysis and feedback as described above.

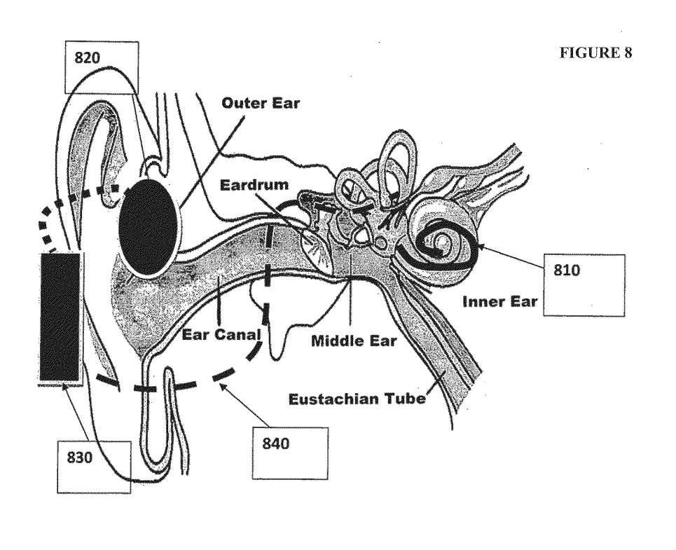

FIG. 8 shows how the device might be used to treat neurosensors deficiencies and hearing impairment. FIG. 8 shows how the device might be used to treat neurosensors deficiencies and hearing impairment. The cochlear implant 810 as described above is implanted in the cochlea. A sound interceptor (for example a microphone) 820, can for example, be placed in the opening of the ear—similar to the placement of a conventional hearing aid. The sound interceptor 820 and the implants are powered by a power source, for example a battery, 830. The battery can also power the other components of the device. The box 830 may also include the microprocessors and other out-of the-ear, external components of the device. The box 830 may, for example, be placed behind the ear, or be carried in a pocket or a pouch in other places or other convenient locations on the user body. The power source and external box 830 may be coupled through the connector 840 to the implant 10. Alternatively or additionally, the box and external power source 830 may be coupled wirelessly to implant 810.

Brain

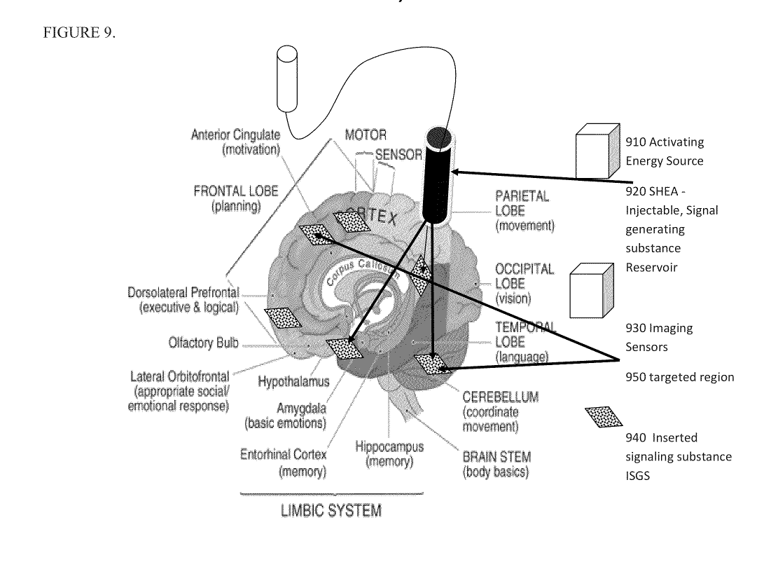

Under some circumstances it become useful to modify and/change brain functions or the functioning of at least some brain components inside the human brain. For example, it may be useful to stimulate with electrical pulses a region of the brain known as area 25 for treatment against depression. FIG. 9 shows additional exemplary areas of an exemplary brain target and the functional regions associated with said brain regions.

Other areas of the brain are responsible for epilepsy and research have shown that selective removal or modification or stimulation (or a combination of these actions) may be beneficial in treating such epileptic conditions as well as Parkinson disease, MS, Alzheimer and other possible brain conditions.

The embodiments disclosed herein contemplate inducing modification in regions of the brain without the need to breach the brain protective skull and without leaving conduits to the out-of the skull or out of the body environment such as leads that conduct electricity to stimulating electrode.

The embodiments disclosed herein contemplate the use of external energy sources such as: Electromagnetic energy, Ultrasound, microwave energy, RF energy, light energy, thermal energy, mechanical energy sources, x-rays, magnetic energy, electric energy, chemical energy, or other forms of energy.

A conduit to the Region-to be Excited (RtbE) for example electric leads, or electrodes, optical fibers, hollow waveguides, or other conduits of energy may be place inside the brain at some distance below the skull surface, and leading to the RtbE.

The energy source is placed outside the skull (for example at a shirt pocket, in a purse, or in other places sufficiently close or at least capable of transmitting signal from the energy source to the leads that were placed inside the skull.

Alternatively or additionally, stimulating members such as electrode, fluorescence dyes, ultrasound source, light sources, mechanical transducers, microphones or other stimulating source may be placed directly near or at the RtbE, or a Region to be Stimulated (RtbS) and said external energy source is then used to excite the targeted region or stimulate the RtbS, so that treatment of the area occurs without the need to breach the surface of the skull or the protective layers of the skull.

As a preventive measure for unwanted brain activity or illnesses or diseases of the brain, a device or a substance or medicine or drugs can be inserted into the brain and deposited in various regions, and then activated when needed, manually or automatically. Such activation can provide preventive treatment, preemptive treatment, therapeutic treatment, imaging, sensing, mapping or storing information.

Additionally or optionally, an imaging or monitoring methods may comprise a device or a drug or a substance can be inserted into a targeted region, and activated when needed, manually or automatically to provide enhanced imaging.

Definitions:

In the context of the embodiments disclosed herein, the word conduit, in addition to its normal meaning used in the art and in the English language, also means channel, canal, duct, passage, pipe, a fiber, a tube, a hollow guide, or any other means to deliver a fluid, liquid, solid, gas, or plasma (physical plasma or biological plasma) to a targeted location within the brain.

In some embodiments, the method includes inserting a substance or a member that is capable of responding to an external signal. The embodiments described herein below will show at least some of the details of the structure and design of the substance or member capable of responding to an external signal.

A Substance of Energy Absorption (SEA) will be referred to as SEA. A SEA is a substance capable of absorbing at least some of a willfully activated energy directed towards a targeted region of the brain or targeted region of a tissue.

In some embodiments, a substance capable of responding to willful energy signal shall be called an Energy Responsive Substance (ERS).

In some embodiments the method comprises, of the steps of providing an energy source capable of exciting a substance. The energy source can be placed outside the body, (for example on a clothing article), or alternatively or additionally inside the body, (for example implanted inside the body, under the skin, etc.).

The method further comprises inserting a substance, ERS, capable of being excited by the energy source into a region of the brain where stimulation, excitation or a response is desired.

The method may further comprise introducing a substance that can be excited by an external energy source. ERS, into the targeted tissue and exciting said substance by a light source. The ERS substance may comprise a light-excitable substance, for example, a photosensizer.

In further embodiments, the ERS may be a photosensitizer which can be excited from a ground singlet state to an excited singlet state and then undergoes intersystem crossing to a longer-lived excited triplet state.

For example, in some embodiments the ERS may be photosensitizer in the proximity of oxygen. When the ERS photosensitizer is in the proximity of an oxygen molecule are in proximity, an energy transfer can take place that allows the photosensitizer to relax to its ground singlet state, transferring energy to create an excited singlet state oxygen molecule. Singlet oxygen is a very aggressive chemical species and will very rapidly react with any nearby bimolecular.

### EXAMPLE OF PHOTOSENSITIZERS

A wide array of photosensitizers for PDT exist. They can be divided into porphyrins, chlorophylls and dyes.[24] Some examples include aminolevulinic acid (ALA), Silicon Phthalocyanine Pc 4, m-tetrahydroxyphenylchlorin (mTHPC), and mono-L-aspartyl chlorin e6 (NPe6).

Several photosensitizers are commercially available for clinical use, such as Allumera, Photofrin, Visudyne, Levulan, Foscan, Metvix, Hexvix, Cysview, and Laserphyrin, with others in development, e.g. Antrin, Photochlor, Photosens, Photrex, Lumacan, Cevira, Visonac, BF-200 ALA. Amphinex. Also Azadipyrromethenes.

Although these photosensitizers can be used for wildly different treatments, they all aim to achieve certain characteristics: High absorption at long wavelengths, High singlet oxygen quantum yield, Low photobleaching, Natural fluorescence, High chemical stability, Low dark toxicity, and preferential uptake in target tissue. High absorption at long wavelengths is desired since tissue is much more transparent at longer wavelengths (˜700-850 nm). Absorbing at longer wavelengths would allow the light to penetrate deeper, and allow the treatment of larger tumors. Natural fluorescence is desired since many optical dosimetry techniques, such as fluorescence spectroscopy, depend on the drug being naturally fluorescent. Low dark toxicity is desired since the photosensitizer should not be harmful to the target tissue until the treatment beam is applied.

The major difference between different types of photosensitizers is in the parts of the cell that they target. Unlike in radiation therapy, where damage is done by targeting cell DNA, most photosensitizers target other cell structures. For example, mTHPC has been shown to localize in the nuclear envelope and do its damage there. In contrast, ALA has been found to localize in the mitochondria and Methylene Blue in the lysosomes.

Since the embodiments disclosed herein contemplate manual or willful insertion of ERS into targeted brain or tissue location, Photosensitizer used with some of the embodiments may be simply injected or inserted directly into the targeted region, without the need for tissue-specific or cell-specific uptake.

“Targeted material” is the targeted tissue or material, which is the target of the treatment or imaging desired by the operator. For example, the targeted material may be brain tissue, the heart, or even non biomaterial, such as implanted metal joints, or inorganic material that one may wish to treat.

“Targeted region” is the region targeted by the method or device described by the present invention as the desired volume of the material, designated for treatment, modification, or imaging.

For example a region of the lens of the eye can be such targeted region. For example, a segment or a portion of a volume of the brain stem can be such targeted region. For example, as shown in FIG. 9 a segment, or a portion, or a partial volume of the Hypothalamus, can be such targeted region. Details of a suggested device and method are shown in FIG. 9.

“Interaction zone” is defined herein as the zone in which the beam of light, a laser beam, or any other energy beam, or energy radiation is interacting with directly (i.e. the interaction zone is the region or volume that effect directly the tissue, for example, photons coming from the energy source (even if said photons underwent a scattering event), being absorbed or otherwise interacting with tissue components in the targeted tissue or targeted region.

The interaction zone defined herein does not include zones or volumes that are not affected directly by the willfully activated energy signal. I.e. the interaction zones does not include regions of collateral damage or regions affected or effected by secondary signals, for example, secondary signals coming from the ERS substance in its capacity to respond to the willfully triggered energy source.

Additional ERS substances that may be used in embodiments of the present invention are described in the provisional patent application Ser. No. 61/252,471, filed Oct. 16, 2009 and entitled: A method and a device for Substance Delivery into Tissue and Skin and Tissue Alterations, incorporated by reference herein.

Nanoparticles

Further, in an additional embodiment an ERS comprise nanoparticles.

Nanoparticles are inserted into the subject brain or body (for example, directly to a brain location or targeted tissue or by injection into to the blood circulation through IV injection, or through digestion by mouth. The nanoparticles arrive to regions in close proximity to the tumor and a process of conjugation of the nanoparticles takes place according to the characteristic pharmacokinetic profile. Thus, the targeted brain region or tissue cells bond to nanoparticles through strong chemical bonds configured as antigen-antibody complex.

Previous studies have shown that Nanoparticle can be delivered to specific locations within the body through the use of the natural immune system. Conjugation of nanoparticle to antibody can then be used for targeting of antigen. For example, antibodies, can be bound to nanoparticles through adhering polymers, (For example PEG) so that antibodies are transferred to tumors or other desired targets by T-Cells or other components of the immune system.

Biocompatible magnetic nanoparticles are able to respond to EM radiation, and to time-varying magnetic fields. Thus nanoparticles that respond to EM radiation, electric fields or magnetic fields can comprise such SHEA.

Additional examples of materials that can serve as SEA are iron oxides. For example Fe₃O₄, Gama-Fe₂O₃, MO—Fe₂O₃, (where M can be Mn, Co, Ni, Ni, or Cu). These iron oxides display ferrimagnetism. Magnetite (Fe₃O₄) Meghemite (Gama-Fe₂O₃) and hematitie (Alpha-Fe₂O₃) are the most common iron oxides.

Another SEA are magnetic Nanoshells. Nanoshells may comprise an iron core with a diameter of about 8 nm coated with a layer of gold. The gold coating is useful in preventing oxidation and enhances biocompatibility. The Gold or other noble metal coatings may be as thin as about 2 nm.

Nanoparticle core can also be coated with block copolymer stabilizers that are compatible with analgesics to prevent flocculation in the arterial system in vivo. Block copolymers also assist in preventing agglomeration. Other possible nanoparticle materials include platinum compounds, vanadium oxides, cobalt, nickel, lanthanum, and manganese.

If the ERS comprises (at least in part) particles, for example nanoparticles, the size of the particles is an important parameter to be considered.

For example, specific absorption rate values of aqueous suspensions of magnetite particles with different diameters varying from 7.5 to 416 nm were by measuring the time-independent temperature curves in an external altering magnetic field (80 kHz, 32.5 kA/m). Results indicate that the SAR values of magnetite particles are strongly size dependent. For magnetite larger than 46 nm, the SAR values increase as the particle size decreases where hysteresis loss is the main contribution mechanism. For magnetite particles of 7.5 and 13 nm which are superparamagnetic, hysteresis loss decreases to zero and, instead relaxation losses (N′eel loss and Brownian rotation loss) dominate.

Additionally, the density of particles or density of a SEA solution must be large enough to create the desired therapeutic or diagnostic/imagine effect, for example generation of a desired thermal or chemical effects, or generation of Electric, magnetic or EM signal, yet be low enough to avoid toxic reaction by the body or tissue or other negative bio-effects. Toxicity, pharmacokinetics, clogging, pollution, clearing from the body and tissue environment must also be consider, along with other negative effects should be considered as well.

Finally, coating of nanoparticles (e.g., derivatives of dextran, polyethylene glycol (PEG), polyethylene oxide (PEO), and poloxamers and polyoxamines) and suspending medium also influence the signal, heat, or chemical effects that the operator may want to create inside the brain tissue or other tissue in the body. The frequency of application, the period of time of excitation field application and duration (e.g., continuous, pulsed) also affects the SAR which is proportional to the power dissipate.

Additionally, another parameter to consider is the presence of nanoparticles' agglomeration and its effect on signal and thermal, chemical, mechanical, electric, magnetic, and other biophysical effects in and on the targeted tissue.

FIG. 9 shows some of the component of the device for treating brain or tissue targets as well as some of the possible targets within a human brain.

For example, an activating energy source 910 can be placed or worn on the body (Or possibly, in some embodiment, implanted or placed inside the body). Alternatively or additionally such energy source may also be located at the operating rooms, outpatient clinics, or as a home device.

Similarly an imaging/sensing member 930 may also be worn on the body (Or possibly, in some embodiment, implanted or placed inside the body). Alternatively or additionally such imaging or sensing source may also be located at the operating rooms, outpatient clinics, or as a home device.

An injected or inserted substance capable of responding to the external energy source by generating or creating its own response 940 may be injected or inserted through a conduit 920 into the desired or intended targeted volume, 950.

The external energy source may, for example, comprise an electric field source, and Electromagnetic energy source, a thermal energy source, an acoustic energy source, a mechanical energy source.

A device or a method of the present invention may further comprise activating a lower power energy source in combination with sensors to achieve imaging or monitoring of the targeted regions.

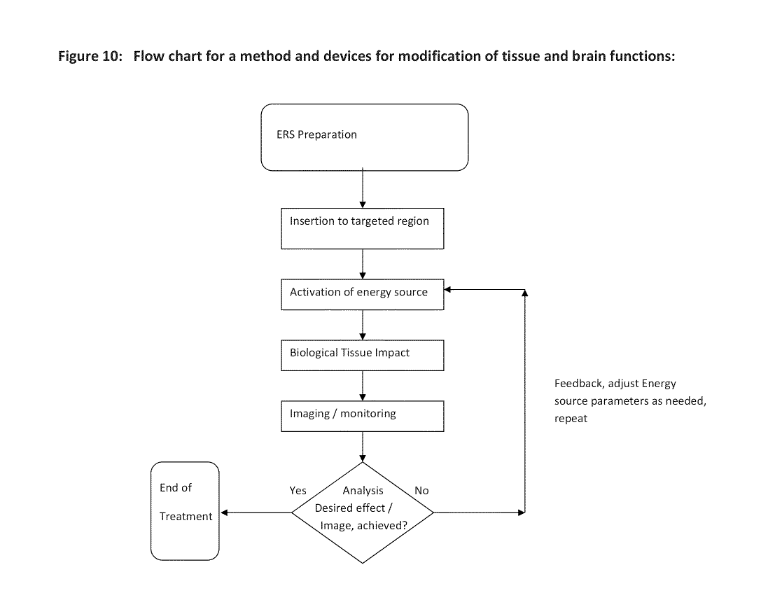

FIG. 10 shows a schematic representation of a method of the present invention. A substance with high absorption properties or properties that allow it to absorb at least some of the willfully triggering energy (SEA or ERS) is prepared and then inserted into the targeted tissue or brain.

At the desired moment the energy source is activated. For example, the energy source may be activated by an operator, by a patient or subject of the treatment or by an automated system, for example, a microprocessor, a computer controller, or other automated controllers configured to monitor body, brain, organ, or tissue functioning and equipped with the software and programs to make decision as to the need for activation.

Upon activation of the of the energy source the energy absorbed or detected by the SEA either creates a biophysical effect (bio impact) or induce the SEA to emit its own signal or its own energy in response to the willfully triggered signal from said first energy source.

The biophysical impact may, for example, comprise, heating of the brain tissue, organ or other body tissue, vaporizing the targeted volume, expending the volume of the targeted volume, modifying the chemistry of the targeted tissue, modifying the physical properties of the targeted volume, modifying the porosity of the targeted volume, or creating other modifications, permanent, temporary, or transient, of the targeted tissue.

Targeted tissue properties as well as time and space dependent properties of the tissue can be monitored, before, during and after the activation and action

Targeted tissue properties can be monitored, for example, optically, thermally, magnetically, with x-ray, with functional MRI, with MRI, with ultrasound, or other imaging means known in the art or imaging ad sensing means described in this specification.

As an example of an impact of the SEA triggered by the energy source, stimulation of nerve or neurons in the brain or other location in the body may be considered. For example, by heating the neuron or changing the rate of thermal energy deposition of the neuron, one can achieve activation of nerves or modification in the state of the neuron or nerves.

Another example may be stimulation of neuron or hair in the cochlea of the ear. For example a SEA material can be placed at various locations along the cochlea and the SEA in each location may be design to respond to different signals from the energy source. As a non-limiting example, consider a magnetic field or an EM field or RF source, or electric signal, or electric wave, or ultrasound signal or vibrational signal, or thermal signal from the energy source, wherein said signal is modulated to carry information for a particularly encoded SEA along a region of the cochlea (or for that matter, again, as a non-limiting examples, the optical nerve, optical receptors, emotional region, memory locations etc.)

For example if a particular SEA is designed to comprise with a particle size/diameter, X, wherein a mechanical or magnetic excitation with frequency W is arriving from the external source, such that W is a resonance frequency of said particle X, only particle X will response to generate a signal or for example release a drug or stimulant. Thus only the region where X is located will be stimulated.

We can thus see that a selector/interceptor outside the body, can intercept sound, or lights, or visions, decompose the incoming information (picture, or speech etc.) much as is done in digital cameras or in digital sound recording, store it in digital storage media, for later use, or transmit it through coded energy signals (electric, electronic, EM signal, magnetic Signal, Ultrasound, microwave, RF, etc.) to the SEA placed in specific desired locations within the tissue or brain, for activation by said incoming signal, of only the specific type of SEA designed for a given particular stimulation.

Additionally or optionally, the impact is monitored by sensors or imaging systems such as CAT, MRI, fMRI, OCT, Encoded fibers, SEE, Ultrasounds, PET, or other imaging and sensing method, to show the impact of the SEA activation on the brain or other body tissue.

The information from the imaging and sensing members can then passed on to an analyzer's and a processor for evaluation (or for manual or human evaluation) and if additional action is needed, as is shown in FIG. 10, the processor instruct the energy source to continue treatment, or to modify treatment parameters and then continue treatment. The process then continues until the desired result is achieved (or until the operator, or the program uploaded to the processor determine that for some reason the desired results or desired result range cannot be achieved, or should not be achieved) and instruct the device to end the process.

As shown in FIG. 10, an exemplary method may comprise of the following steps:

Prepare the ERS in a manner that allows it to respond to external stimulant.

For example, ERS preparation may comprise inserting a substance that can respond to external stimulant (for example, an external EM field, external magnetic field, external electric field, Ultrasound signal, thermal signal, mechanical signal, mechanical deformation, stress or strain, etc.) ERS response may comprise generating EM signal, or by generating for example, an external EM field, external magnetic field, external electric field, Ultrasound signal, thermal signal, mechanical signal, mechanical deformation, stress or strain, etc.)

As a non-limiting example, consider an ERS comprising an electric or magnetic dipole material. For example gold or other metallic nanoparticle. Or, for example, a biocompatible material containing such metallic nanoparticle. In one embodiment the external energy source create heating in the ERS which allows expansion destruction of a targeted tissue. In another embodiment the external energy source heating of the ERS allows expansion of encapsulated members which then release a tissue-modifying substance. In another embodiment the external energy source heating of the ERS allows expansion of encapsulated members which then release a tissue-killing substance. In another embodiment the external energy source is absorbed more efficiently in the ERS allowing heating of the ERS. In another embodiment the external energy source is absorbed more efficiently in the ERS causing the ERS to emit energy after or during the absorption of the external energy. In another embodiment at least some energy is absorbed by the ERS causing the ERS to emit energy after or during the absorption of said energy. The energy emitted by the ERS may then create a desired effect on a region of a targeted tissue near the ERS location. The energy emitted by the ERS may then create a desired effect on a region of a targeted tissue.

Such desired effect may be suppression of depression, suppression of fear, suppression of hunger, changes in mood, faster learning, treatment of epilepsy or other neural stimulation or targeted tissue stimulation or tissue modification or tissue stimulation effects. ERS may be Inserted into a targeted region by placing a predetermined amount of ERS substance into the targeted region.

Obtaining a Desired Effect or Image or Sensing Information.

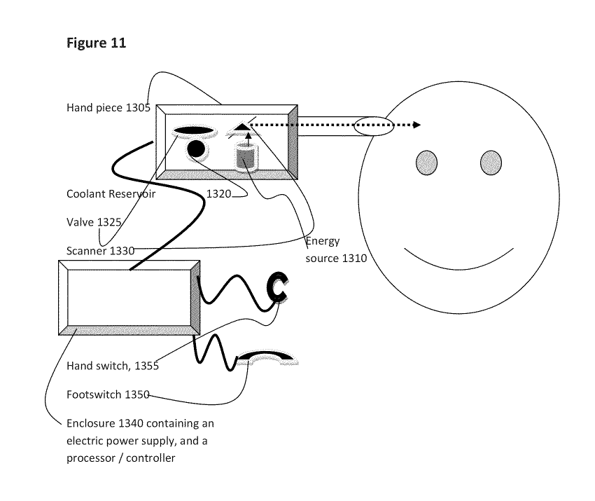

FIG. 11 shows another embodiment of the present invention. An energy source, for example a laser or other EM or magnetic, or electric energy source, is activated. The energy is directed into the skull. Energy travels through the bone skin, bone and other external structure of the skull and reaches the brain to stimulate blood flow and neural activities.

As a non limiting example, in FIG. 11 a laser diode is shown. It emits continuous wave light at about 810 nm. A hand piece 1305 in this exemplary case, may comprise a laser source (or other energy source 1310), a coolant reservoir 1320 with emission valve 1325 for example a fuel injection valve, to emit coolant and cool down the surface of the tissue, a scanner 1330 to move the beam in a predetermined pattern.

An electric power source and microprocessor that control the operation of the device are contained in an exemplary box 1340 and electrically connected to the handpiece.

For example, an Enclosure 1340 containing an electric power supply, and a processor/controller. The enclosure is connected to a footswitch 1350 or hand switch 1355, for willful activation of the device. The footswich or handswitch may be used to activate the device, alternatively, the device may be activated by neurotransmitters or other forms of energy within the body, neural system, circulatory system, or immune system.

A scanning rate of from about 1 cm² per 0.01 sec to about 1 cm² per 100 sec may be used. A scanned are of from about 0.01 cm² to about 100 cm² may be used. The Laser Beam spot size may be from about 0.001 mm in diameter to about 10 cm in diameter, and more preferably from about 0.01 cm in diameter to about 3 mm in diameter.

Once the scanned area have been completely treated, the handpiece may be moved to a new treatment area on the surface of the skull. Some, non-limiting examples of preferred target areas are the frontal lobe, the temporal lobes, the top of the skull, treatment along birth-fractures (or suture lines) in the skull, as well as other areas of the skull.

In a further embodiment of the present invention, the energy source comprises: Mechanical energy, Energy applied to needles to drive said needles into the brain tissue, Energy applied to needles to drive said pins into the brain tissue, Energy applied to other members capable of penetrating tissue to drive said members into the brain tissue, Energy applied to syringes to drive said syringes into the brain tissue, Arrays of the above, Light Energy, Radio Frequency Energy, Electric Energy, Magnetic energy, Chemical energy, microwave energy, ultrasound energy, sound energy, vibration energy, ionizing energy, energy from nuclear decay, Proton beam, Electron Beam, UV energy, X-ray energy, Chemical Energy, Laser energy, Pulse laser energy, Pulse Electromagnetic energy, Pulse electromagnetic energy with pulse duration, Pulse Duration, Pulses shorter than about, 10 minute, 1 minute, 10 sec, 1 sec, 100 ms, 10 ms, 1 ms, 100 microsecond, 10 microsecond, 1 microsecond, 100 nanoseconds (ns), 10 ns, 1 ns, 100 ps, 10 ps, 1 ps, 100 fs, 10 fs, 5 fs.

In a further embodiment of the present invention, the substance capable of interacting with said external energy comprises at least one of the following materials: substance capable of responding to external energy and is biocompatible, nanoparticles, substance capable of absorbing said external energy, Substance capable of absorbing external energy and comprise biocompatible material, carbon-based particles, micro particle, microparticle containing releasable substance, substance containing electric dipole, micro nanoparticles, gold nanoparticles, Silver nanoparticles.

In another embodiment of the present invention, the energy source is mounted on the human body. In another embodiment of the present invention, the energy source is placed inside the human body. In another embodiment of the present invention the ERS substance capable of interacting with said external energy comprises at least one of the following materials, a substance capable of responding to external energy and is biocompatible, nanoparticles, a substance capable of absorbing said external energy, a substance capable of responding to a willful signal carrying energy, a Substance capable of absorbing external energy and comprise biocompatible material, A carbon-based particles, A micro particle, A microparticle containing releasable substance, A substance containing electric dipole, Microparticles, Nanoparticles, gold nanoparticles, Silver nanoparticles

In another embodiment the energy source is mounted on the human body. In yet another embodiment the energy source is located within the body. In another embodiment the ERS substance is capable of creating energy emission in response to a signal from said energy source.

In another embodiment the ERS substance comprise at least one electric dipole. In another embodiment the ERS substance comprise at least one magnetic dipole. In another embodiment the energy delivery to a targeted tissue or region of the brain is enhanced through an intermediate substance. In another embodiment the energy delivery to the targeted tissue or region of the brain is enhanced through an energy conduit substance. In another embodiment at least some tissue surrounding the brain is removed. In another embodiment at least some tissue surrounding the brain is removed and at least some of said removed tissue is at least partly replaced by a substance which allows better transport of energy into the targeted region. In another embodiment the activation of the energy source is accomplished manually. In another embodiment the activation of the energy source is a willful activation by a person whose brain or other portion of his body tissue is being treated.

In another embodiment the activation of the energy source comprises an automatic activation. In another embodiment the activation of the energy source comprises an automatic activation without willful human intervention at the time of said activation. In another embodiment the activation comprises activation by a processor upon analysis of feedback from the treated tissue or brain. The feedback from said tissue or said brain region is generated by sensors and monitoring members capable of monitoring brain function. In another embodiment the activation of energy source comprises activation based on monitoring brain activity.

In another embodiment the energy generated by the ERS in response to the source energy signal is one or more of the following: Thermal energy, Electrical Energy, Magnetic, Mechanical Energy, EM energy, Light Energy, Radio Frequency Energy, Electric Energy, Magnetic energy, Chemical energy, microwave energy, ultrasound energy, sound energy, vibration energy, energy from nuclear decay, Proton beam, Electron Beam, UV energy, X-ray energy, Gamma rays, Alpha Rays, Beta rays.

In another embodiment the excitation energy is internal energy (internal to the body).

—Device—

Additional embodiments of the present invention (See below) discuss devices or apparatus suitable for the practice of the present invention.

In one such embodiment a device is envisioned for the purpose of interacting with a brain component:

In this embodiment, the Device comprises, An energy source capable of exciting a substance; A conduit for inserting a substance capable of being excited by an energy source into a region of the brain where stimulation, excitation or a response is desired; A member capable of activating said energy source at a desired time; and A member capable of monitoring a desired effect or image or sensing information from said substance.

In another embodiment the device described above include an energy source comprises one of the following energy source: Mechanical energy, Energy applied to needles to drive said needles into the brain tissue, Energy applied to needles to drive said pins into the brain tissue, Energy applied to other members capable of penetrating tissue to drive said members into the brain tissue, Energy applied to syringes to drive said syringes into the brain tissue, Arrays of the above, Light Energy, Radio Frequency Energy, Electric Energy, Magnetic energy, Chemical energy, microwave energy, ultrasound energy, sound energy, vibration energy, ionizing energy, energy from nuclear decay, Proton beam, Electron Beam, UV energy, X-ray energy.

In another embodiment the device described comprise an energy source which is a laser emitting a wavelength in one or more of the following approximate ranges: From about 0.4 μm to about 0.8 μm, 0.8 to 1.5 μm, 1.5 to 2.3 μm, 0.2 μm to 0.8 μm.

In yet another embodiment the device's energy source comprises a broadband light emitter, emitting light with wavelength in the range of one or more of the following approximate ranges: 0.8 to 1.5 μm, 1.5 to 2.3 μm, 0.2 μm to 0.8 μm.

In yet another embodiment the device's conduit for inserting a substance into the brain comprises one or more of the following conduit for delivery of a ERS substance: a needle, a hollow tube, a syringe, a hollow waveguide, a pill or oral delivery drug delivery, A topical substance delivery, a scalpel, a catheter, or a suction or pressurized conduit.

In yet another embodiment, the targeted regions of interest for the device stimulating response may include one or more of the following locations: any location within the brain, Any location within the nervous system, any location along the spinal cord, any location in and around about locations where flow of the body natural nervous signaling or electrical signaling or chemical signaling has been interrupted, or any other body locations where stimulation is needed or desired.

In another embodiment, the device further comprises a member capable of activating said energy source. The member capable of activating said energy source may comprise a manual switch operated by the person whose brain or other organ is treated.

An additional embodiment of the device may comprise a computer, processor, CPU, or a processing member which as a non-limiting examples, are capable of activating the device energy source.

An additional embodiment may include a device comprising a member capable of activating said energy source which may include one or more of the following: a computer, cpu, controller, a processor, an automated member acting in response to input from sensors, imagers, or other components capable of providing feedback, information, or tracking and sensing input to the CPU, computer, or controller.

Another embodiment of the preset invention may comprise a member of the device capable of activating said energy source wherein the member is a manual switch operated by a person who is treating the person in need of treatment or in need of surgery.

Yet another embodiment of the preset invention may comprise a member capable of activating said energy source, said member comprises a manual switch operated by a person who is treating the person in need of treatment or in need of surgery, for example, a doctor, a physician, a nurse, a health care professional, or other person who is providing said treatment to said person being treated.

Another embodiment of the preset invention may comprise an energy source which include at least one of the following energy sources: Mechanical energy, Energy applied to needles to drive said needles into the brain tissue, Energy applied to needles to drive said pins into the brain tissue, Energy applied to other members capable of penetrating tissue to drive said members into the brain tissue, Energy applied to syringes to drive said syringes into the brain tissue, Arrays of the above, Light Energy, Radio Frequency Energy, Electric Energy, Magnetic energy, Chemical energy, microwave energy, ultrasound energy, sound energy, vibration energy, ionizing energy, energy from nuclear decay, Proton beam, Electron Beam, UV energy, X-ray energy.

FIG. 9 shows a schematic diagram of the brain and the major brain components. The invention comprises a substance that can be activated by an external radiation. The FIG. 9 shows such external radiation source. The substance is injected into the targeted location in the brain. Activation of the external radiation willfully stimulates the substance injected into the brain and creates a response in said injected substance (IS), for example a secondary electric or magnetic field or EM fields or E or Magnetic or EM radiation. Said stimulated secondary radiation such as an ERS discussed above, to create a localized stimulation of a desired region of the brain. Alternatively, the injected substance or ERS is stimulated to release a drug or a chemical or create heat or generate a secondary effect or release of stimulant or create another physical, chemical, biophysical, or biochemical desired effect in the targeted region.

Definition:

In the context of the present invention, the word conduit, in addition to its normal meaning used in the art and in the English language, also means channel, canal, duct, passage, pipe, a fiber, a tube, a hollow guide, or any other means to deliver a fluid, liquid, solid, gas, or plasma (physical plasma or biological plasma) to a targeted location within the brain.

An embodiment of the present invention may include a device for interacting with a brain or tissue component wherein the device utilizes one or more of the method embodiments described above.

An embodiment of the present invention may include a method for interacting with a brain or tissue component wherein the method utilizes one or more of the device embodiments described above.

Additional embodiment of the present invention may comprise a method for interacting with a brain component wherein the method comprises the steps of: identifying a person to be treated; applying one or more of the device embodiments described above.

Further embodiment of the present invention may comprise a conduit for inserting an ERs substance capable of being excited by the energy source comprises a small diameter pipes, tubes, hollow guide, needle, syringes, or other conduit capable of penetrating the brain tissue substantially without causing significant damage to the brain tissue.

Further embodiment of the present invention may comprise a conduit for inserting a substance capable of being excited by the energy source, wherein said ERS delivery conduit is inserted into the brain and directed to the target region by a mechanical force.

In an additional embodiment of the present invention, the conduit for inserting a substance capable of being excited by the energy source, is inserted into the brain and directed to the target region while directed through an imaging, and/or sensors, providing feedback and guidance to as to the rout taken by the conduit.

Another non-limiting exemplary embodiment may comprise a method for the treatment and surgery of the brain include:

a) Introducing a substance that is responsive to an external energy field to the region of the targeted material (for example a human brain may be the targeted material and the hypothalamus may be the targeted region. Or a specific section of the hypothalamus may be the targeted region).

b) Activating or modulating said introduced substance by an external energy field at the desired time and with the desired intensity of the external field. For example, an external stimulating field may be a magnetic field (for example, like the one used in MRI devices), an electric field, an electromagnetic (EM) field, an EM radiation, a laser radiation, a radiofrequency (RF) radiation, Terahertz radiation, microwave radiation, heating energy, cooling and removal of energy, or other components of the EM spectrum, an ultrasound energy, mechanical energy, thermal energy, chemical energy, nuclear energy or other energy sources capable of external stimulation.

Treatment targets may include: Motion control centers, or Epileptic control centers.

In another embodiment, a method for modifying brain tissue or other targeted tissue wherein a micro needle or hollow wave guide (HWG) of an inner diameter of about 2 mm, 1 mm 0.5 mm 0.1 mm 0.05 mm and 0.001 mm (10 μm) is brought into contact with a targeted tissue or brain tissue, wherein sad hollow wave guide or needle is surrounded by support tube.

In yet another embodiment, the method can further comprise the use of a microneedle or a HWG can deliver USPL pulses that drill substantially with no thermal or mechanical damage and lead to the targeted region, and wherein inner diameter of the HWG or micro-needle is about 10 micrometer or from about 10 micrometer μm to about 50 micrometer. HWG or Microneedle of this size can thus be substantially on the order of the size of a single cell.

Ultrashort pulse laser radiation (for example, with pulse duration from about 3 fs to about 500 ps) can complete the drilling into the targeted tissue region. At that point, the laser or electromagnetic (EM) radiation is stopped and the HWG or micro needles can be used to deliver a desired ERS substance or SEA substance to the targeted region.

In a similar way in the treatment of skin tissue for skin and for fat removal a device and a method as described above can be used to introduce micro needle or HWG into the targeted tissue region and then induce light or EM radiation damage, or drilling, and then said HWG or micro needles can be used to insert a dissolving substance, Botox, toxin, dissolving substance, or other ERS or other substances into the targeted tissue to treat, modify or remove fat, tissue or treat other skin or tissue ailment.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method for modifying brain tissue or other targeted tissue, comprising:
a. Applying a micro-needle or hollow wave guide to a targeted region, wherein the micro needle or hollow wave guide (HWG) includes an inner diameter of about 2 mm, 1 mm 0.5 mm 0.1 mm 0.05 mm and 0.001 mm (10 gm), wherein said hollow wave guide or needle is surrounded by support tube;
b. Creating a conduit by removing tissue so that a substance may be delivered into the targeted tissue; and
c. Applying an energy source to the targeted tissue to obtain a desired effect.

2. The method of claim 1, wherein said microneedle or HWG can deliver USPL pulses that drill substantially with no thermal or mechanical damage and lead to the targeted region, and wherein inner diameter of the HWG or micro-needle is about 10 micrometer or from about 10 micrometer μm to about 50 micrometer.

3. A method for interacting with a brain, comprising:
a. Providing an external energy source capable of exciting a substance;
b. Inserting a substance capable of being excited by the external energy source into a region of the brain where stimulation, excitation or a response is desired;
c. Activating said external energy source at a desired time; and
d. Obtaining a desired effect or image or sensing information.

4. The method of claim 3, wherein the external energy source is selected from a group consisting essentially of: a mechanical energy, an energy applied to needles to drive said needles into the brain tissue, an energy applied to needles to drive said pins into the brain tissue, an energy applied to other members capable of penetrating tissue to drive said members into the brain tissue, an energy applied to syringes to drive said syringes into the brain tissue, a Light Energy, a Radio Frequency Energy, an Electric Energy, a Magnetic energy, a Chemical energy, a microwave energy, an ultrasound energy, a sound energy, a vibration energy, an ionizing energy, an energy from nuclear decay, a Proton beam, an Electron Beam, an UV energy, a X-ray energy, a Laser energy, a Pulsed laser energy, a Pulsed Electromagnetic energy, a Pulsed electromagnetic energy with pulses shorter than about 10 minute, 1 minute, 10 sec, 1 sec, 100 ms, 10 ms, 1 ms, 100 microsecond, 10 microsecond, 1 microsecond, 100 nanoseconds (ns), 10 ns, 1 ns, 100 ps, 10 ps, 1 ps, 100 fs, 10 fs, and 5 fs.

5. The method of claim 3, wherein the substance capable of interacting with said external energy is selected from the group consisting essentially of: a substance capable of responding to external energy and is biocompatible, a plurality of nanoparticles, a substance capable of absorbing said external energy, a substance capable of absorbing external energy and comprise biocompatible material, a carbon-based particle, a micro particle, a microparticle containing releasable substance, a substance containing an electric dipole, a micro-nanoparticle, a gold nanoparticles, and a Silver nanoparticle.

6. The method of claim 3, wherein said substance is capable of creating energy emission in response to a signal from said energy source.

7. The method of claim 3, wherein said energy delivery to the enhanced through an intermediate substance or through an energy conduit substance.

8. The method of claim 3, wherein part of the tissue surrounding the brain is removed and at least partly replaced by a substance which allows better transport of energy into the targeted region.

9. The method of claim 3, wherein said activation comprises activation by a feedback, said feedback is generated by sensors and monitoring members capable of monitoring brain function.

10. The method of claim 3, wherein said activation of energy source comprises activation based on monitoring of brain activity.

11. The method of claim 6, wherein the energy generated in response to said signal is selected from the group consisting essentially: a Thermal energy, an Electrical Energy, a magnetic, a Mechanical Energy, an EM energy, a Light Energy, a Radio Frequency Energy, an Electric Energy, a Magnetic energy, a Chemical energy, a microwave energy, an ultrasound energy, a sound energy, a vibration energy, an energy from nuclear decay, a Proton beam, an Electron Beam, an UV energy, an X-ray energy, a gamma ray, an Alpha Ray, or a Beta ray.

12. A device for interacting with a brain component, comprising:
a. An external energy source capable of exciting a substance;
b. A conduit for inserting a substance capable of being excited by the external energy source into a region of the brain where stimulation, excitation or a response is desired;
c. A member capable of activating said external energy source at a desired time; and
d. A member capable of monitoring a desired effect or image or sensing information from said substance.

13. The device of claim 12, wherein the energy source is selected from the group consisting essentially of: a Mechanical energy, an Energy applied to needles to drive said needles into the brain tissue, an Energy applied to needles to drive said pins into the brain tissue, an Energy applied to other members capable of penetrating tissue to drive said members into the brain tissue, an Energy applied to syringes to drive said syringes into the brain tissue, a Light Energy, a Radio Frequency Energy, an Electric Energy, a Magnetic energy, a Chemical energy, a microwave energy, an ultrasound energy, a sound energy, a vibration energy, an ionizing energy, an energy from nuclear decay, a Proton beam, an Electron Beam, an UV energy, and an X-ray energy.

14. The device of claim 12, wherein said energy source is a laser emitting a wavelength in one or more of the following approximate ranges: 0.8 to 1.5 μm, 1.5 to 2.3 μm, or 0.2 μm to 0.8 μm.

15. The device of claim 12, wherein said energy source is a broadband light emitter, emitting light with wavelength in the range of one or more of the following approximate ranges: 0.8 to 1.5 μm, 1.5 to 2.3 μm, 0.2 μm to 0.8 μm.

16. The device of claim 12, wherein said conduit for inserting a substance into the brain is selected from a group consisting essentially of: a conduit for delivery of said substance, a needle, a hollow tube, a syringe, a hollow waveguide, a pill or oral delivery drug delivery, A topical substance delivery, a scalpel, a catheter, and a suction or pressurized conduit.

17. The device of claim 12, wherein the member capable of activating said energy source is selected from the group consisting essentially of: a computer, a cpu, a controller, a processor, an automated member acting in response to input from sensors, imagers, or other components capable of providing feedback, information, or tracking and sensing input to the CPU, computer, or controller.

18. The device of claim 12, where the energy source is selected from the group consisting essentially of: a Mechanical energy, an Energy applied to needles to drive said needles into the brain tissue, an Energy applied to needles to drive said pins into the brain tissue, an Energy applied to other members capable of penetrating tissue to drive said members into the brain tissue, an Energy applied to syringes to drive said syringes into the brain tissue, a Light Energy, a Radio Frequency Energy, an Electric Energy, a Magnetic energy, a Chemical energy, a microwave energy, an ultrasound energy, a sound energy, vibration energy, an ionizing energy, an energy from nuclear decay, a Proton beam, an Electron Beam, an UV energy, and an X-ray energy.

19. The device of claim 12, wherein the conduit for inserting a substance capable of being excited by the energy source comprises a small diameter pipes, tubes, hollow guide, needle, syringes, or other conduit capable of penetrating the brain tissue substantially without causing significant damage to the brain tissue.

20. The device of claim 12, wherein the conduit for inserting a substance capable of being excited by the energy source, is inserted into the brain and directed to the target region while directed by an imaging and/or sensors, providing feedback and guidance to as to the rout taken by the conduit.
